(19) United States
(12) Patent Application Publication
(10) Pub. No.: US 2008/0306273 A1
(43) Pub. Date: Dec. 11, 2008

(54) Process for the Preparation of 1-Aminopiperidine Derivatives

(76) Inventors: Dieter Most, Bruchkobel (DE); Kai Rossen, Hanau (DE)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/885,922

(22) PCT Filed: Feb. 28, 2006
(86) PCT No.: PCT/EP2006/060320

§ 371 Date: Aug. 7, 2008

(30) Foreign Application Priority Data
Mar. 10, 2005 (DE) 102005011050.9

(51) Int. Cl. C07D 295/027; C07D 295/023

(52) U.S. Cl. 546/244

(57) ABSTRACT

The present invention relates to a process for the preparation of compounds of the general formula (I).

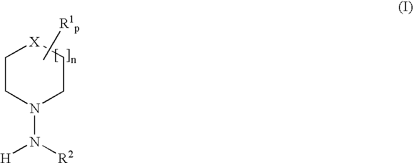

These are used as important intermediates in the preparation of bioactive substances. Preparation is carried out starting from dicarbonyl compounds of the general formula (II),

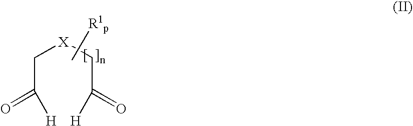

which are reacted with appropriate hydrazine derivatives of the general formula (III)

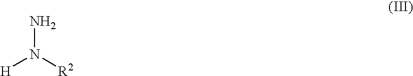

and subsequently hydrogenated. The present invention also relates to advantageous intermediate compounds.

The present invention is aimed at a process for the preparation of compounds of the general formula (I).

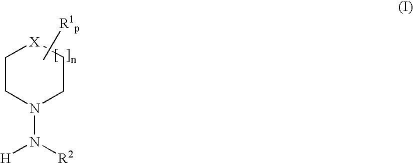

Hydrazine derivatives of the formula (I) are valuable intermediates for the preparation of biologically active molecules. Thus, compounds of the general formula (I) are used in the synthesis of CB₁ antagonists such as, for example, Rimonabant® (EP 656354; Shim et al., J. Med. Chem. 2002, 45, 1447-1459; Lan et al., J. Med. Chem. 1999, 42, 769-776).

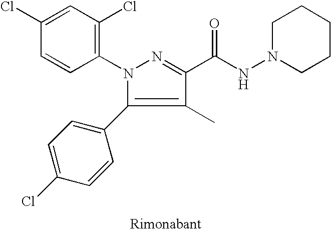

The preparation of, for example, 1-aminopiperidine is familiar to the person skilled in the art. Thus, Auelbekov et al. and Seebach et al. propose reducing 1-nitroso-piperidine to the amino derivative in the presence of Zn/AcOH (Khimiko-Farmatsevticheskii Zhurnal, 1985, 19, 829-32; Synthesis 1979, 6, 423-4). Jain et al. describe the reaction of piperidine with chloramine to give the corresponding hydrazine derivatives (Proceedings—Indian Academy of Science, Chemical Sciences 1985, 95, 381-9).

The proposed synthesis routes allow the preparation of the envisaged compound only using chemicals which cannot be used on the industrial scale without special protective measures as regards apparatus. Thus, the use of a Zn/acetic acid mixture as a reducing agent is disadvantageous on account of the heterogeneity of the reaction and of the excess of Zn which must be employed in the reaction. The work-up of the reaction batch is, as a rule, relatively complicated. The handling of the 1-nitrosopiperidine, which is a very strong carcinogen, also represents a great technical problem. Although chloramine is a widespread reagent for the disinfection of drinking water, its use in concentrated form is questionable for industrial safety reasons. Special safety precautions must guarantee that contamination of the workplace and of the environment by the gas is avoided, since it is damaging to the lungs in relatively high concentration.

The object of the present invention was therefore to specify a further process for the preparation of compounds of the general formula (I). In particular, the process should advantageously be employable on a large scale in comparison with the processes of the prior art. It should moreover be implementable in chemical plants without great expenditure and superior to the known processes from the economic and ecological points of view.

The object is achieved according to the claims.

As a result of starting, in a process for the preparation of compounds of the general formula (I)

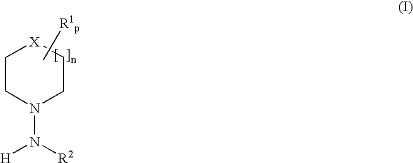

in which
n can be =0, 1,
p can be =0, 1, 2, 3,

### X can be CR¹R¹, O, NR², NR¹, S,

R¹ independently of one another can be H, (C₁-C₈)-alkyl, (C₁-C₈)-alkoxy, (C₁-C₈)-alkoxyalkyl, (C₃-C₈)-cycloalkyl, (C₆-C₁₈)-aryl, (C₇-C₁₉)-aralkyl, (C₃-C₁₈)-heteroaryl, (C₄-C₁₉)-heteroaralkyl, ((C₁-C₈)-alkyl)₁₋₃-(C₃-C₈)-cycloalkyl, ((C₁-C₈)-alkyl)₁₋₃-(C₆-C₁₈)-aryl, ((C₁-C₈)-alkyl)₁₋₃-(C₃-C₁₈)-heteroaryl,
R² is H or an N-protective group which can be cleaved under acidic or basic conditions,
from a dicarbonyl compound of the general formula (II),

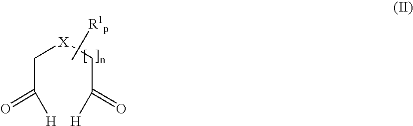

in which X, R¹, n, p can assume the meaning indicated above, and reacting this with one equivalent of the hydrazine derivative of the general formula (III)

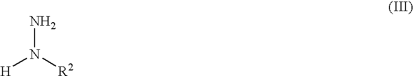

in which
R² is an N-protective group which can be cleaved under acidic or basic conditions,
and subsequently hydrogenating the compound formed in the presence of a transition metal and optionally furthermore performing the cleavage of the group R² under acidic or basic conditions, the object set is achieved extremely surprisingly, but for that no less advantageously. It is possible with the present process to prepare hydrazine derivatives of the general formula (I) in yields of >76% starting from the dicarbonyl compound.

In principle, the person skilled in the art, within the scope indicated, can employ for the synthesis all starting compounds of the general formula (II) or (III) appearing conceivable to him. He orients himself here on the reactivity of the compounds employed and preferably takes those which are capable of entering into the reaction described, but otherwise prove inert under the reaction conditions, in order to suppress the generation of by-products as much as possible during the reaction.

In relation to the index n, the person skilled in the art preferably chooses those compounds of the general formula (II) which form a five- or six-membered ring. The index p is preferably 0. Advantageously, X assumes radicals such as CH₂ or O. R¹ is, in a preferred embodiment, H, (C₁-C₈)-alkyl or (C₆-C₁₈)-aryl. R² is an N-protective group such as formyl, acetyl, propionyl, benzoyl, aryl-, arylalkyl- or alkoxy-carbonyl, such as methoxycarbonyl, ethoxycarbonyl, propoxy-carbonyl, Z, Boc, phenoxycarbonyl.

A process is very particularly preferred in which compounds of the formula (II) or (III) are employed, in which n is =1, p is =0, X is ═CH₂, R² is =acetyl or methoxycarbonyl.

For the hydrogenation, the person skilled in the art can use the transition metals suitable to him for this purpose. These can be employed in the hydrogenation in the form of the known homogeneously soluble transition metal complexes, which contain metals such as Ru, Rh, Pt, Pd as the central atom, or as heterogeneously soluble, optionally supported transition metals. Transition metal complexes preferably to be employed can be found from the literature (Katalytische Hydrierungen im Organisch-Chemischen Laboratorium [Catalytic Hydrogenations in the Organic Chemistry Laboratory], F. Zymalkowski, Ferdinand Enke Verlag Stuttgart, 1965). Very particularly preferably, those catalysts are employed which contain Pt or Pd. Extremely preferred heterogeneously soluble transition metals as a catalyst are Pd/C, PtO₂, Pt/C.

The hydrogenation mentioned can be carried out as a hydrogenation using H₂ gas or as a transfer hydrogenation. These procedures are likewise known to the person skilled in the art (“Asymmetric transfer hydrogenation of C═O and C═N bonds”, M. Wills et al. Tetrahedron: Asymmetry 1999, 10, 2045; “Asymmetric transfer hydrogenation catalyzed by chiral ruthenium complexes” R. Noyori et al. Acc. Chem. Res. 1997, 30, 97; “Asymmetric catalysis in organic synthesis”, R. Noyori, John Wiley & Sons, New York, 1994, p. 123; “Transition metals for organic Synthesis” Ed. M. Beller, C. Bolm, Wiley-VCH, Weinheim, 1998, Vol. 2, p. 97; “Comprehensive Asymmetric Catalysis” Ed.: Jacobsen, E. N.; Pfaltz, A.; Yamamoto, H., Springer-Verlag, 1999). The hydrogen pressure to be set in the reaction according to the invention can be chosen arbitrarily by the person skilled in the art. Preferably, a pressure from 1 to 100 bar, more preferably 1 to 50 bar and very particularly preferably 1 to 30 bar, is set. Extremely preferred here is a pressure range from 1 to 20 bar.

The transition metal complexes mentioned can be employed in the reaction in an amount of from 0.1-10 mol % based on compound (II). Preferably, an amount of from 0.5-7.5 mol %, more preferably 1.0-5.0 mol % and very particularly preferably 2.0-3.0 mol % is employed. The person skilled in the art orients himself in the choice of the amount on the reaction economy, meaning to say that with a yield which is as optimal as possible as little as possible expensive catalyst is employed.

The cleavage of the protective group R² is carried out optionally. It can preferably be carried out in an acidic aqueous or basic aqueous solution. To this end, an inorganic acid is more advantageously dissolved in water or a solution of an inorganic base in water is employed for cleavage of the protective groups. Aqueous solution is understood according to the invention as meaning a homogeneous solution of the inorganic acid or base in water as the main constituent (>50 mol %) of the mixture. Suitable inorganic acids are, in particular, acids such as hydrochloric acid, sulphuric acid or phosphoric acid. Inorganic bases can be selected from the group consisting of alkali metal carbonate, alkali metal hydroxide, in particular lithium hydroxide, sodium hydroxide and potassium hydroxide.

The temperature during the reaction can be between RT and 140° C. Preferably, a temperature range from 80° C. to 140° C. and extremely preferably between 100° C. and 130° C. is set.

The person skilled in the art is free in the choice of whether he would like to carry out the individual reaction steps mentioned sequentially or together in one pot. A process is preferred, however, in which the reaction of the compound of the formula (II) with the compound of the formula (III) and the hydrogenation of the compound formed therefrom is carried out as a one-pot reaction. Optionally, the entire reaction can also be carried out in one pot. According to the invention, entire reaction here is understood as meaning the preparation of the compound of the general formula (III), its reaction with compounds of the general formula (II), the hydrogenation of the intermediate formed and the optional cleavage of the N-protective groups (see Example 2). The compound of the general formula (I) is thus obtained in a manner which is simple and can be carried out particularly readily on the industrial scale.

Suitable solvents for the reaction according to the invention are essentially water, alcohols, ethers or mixtures thereof. Preferably, water in the presence of alcohols (methanol or ethanol) is employed. The reaction can be carried out homogeneously as a single phase or as two phases, the homogeneous procedure, however, being preferred. The work-up of the reaction mixture is carried out according to processes known to the person skilled in the art by distillation, extraction and/or crystallization of the products of the formula (III).

The present invention also relates to intermediate compounds of the general formula (V).

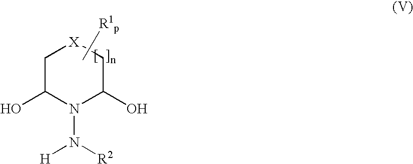

In these, as described further at the front for compounds of the general formula (I), n is =0, 1, p can be 0, 1, 2, 3, X can be CR¹R¹, O, NR², NR¹, S, R¹ is H, C₁-C₈)-alkyl, (C₁-C₈)-alkoxy, (C₁-C₈)-alkoxyalkyl, (C₃-C₈)-cycloalkyl, (C₆-C₁₈)-aryl, (C₇-C₁₉)-aralkyl, (C₃-C₁₈)-heteroaryl, (C₄-C₁₉)-heteroaralkyl, ((C₁-C₈)-alkyl)₁₋₃-(C₃-C₈)-cycloalkyl, ((C₁-C₈)-alkyl)₁₋₃-(C₆-C₁₈)-aryl, ((C₁-C₈)-alkyl)₁₋₃-(C₃-C₁₈)-heteroaryl, and R² is H or an N-protective group which can be cleaved under acidic or basic conditions.

A compound as follows is very particularly preferred:

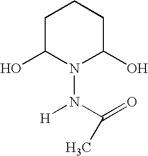

The reaction according to the invention can be carried out by way of example by reacting an aqueous solution of the dicarbonyl compound (II), for example glutaraldehyde, with a compound of the general formula (III), for example acetylhydrazine. The aqueous (glutaraldehyde) solution is treated with the compound (II) optionally dissolved in a solvent such as ethanol and added to an autoclave in the presence of a catalyst (for example 5% Pd/C). After hydrogenation at a hydrogen pressure of 20 bar and a temperature of 80° C., the reaction is as a rule complete after one hour. The compound of the general formula (III) obtained, in this case 1-acetamidopiperidine, can be worked up according to the person skilled in the art and isolated by distillation.

Subsequently, the cleavage of the N-protective group can be carried out as indicated. The work-up of the cleavage solution is preferably carried out by separation of the phases and extraction of the reaction mixture with organic solvents suitable for this purpose to the person skilled in the art. These are subsequently combined and the compound of the general formula (III) where R²═H is isolated from them, for example, by distillation. The total yield of the reaction described here is >76%.

The following reaction schemes illustrate the procedure described again:

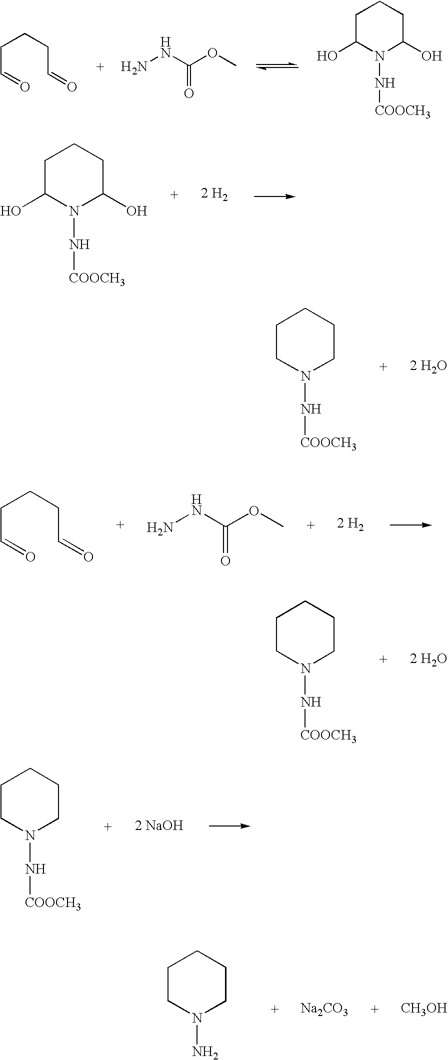

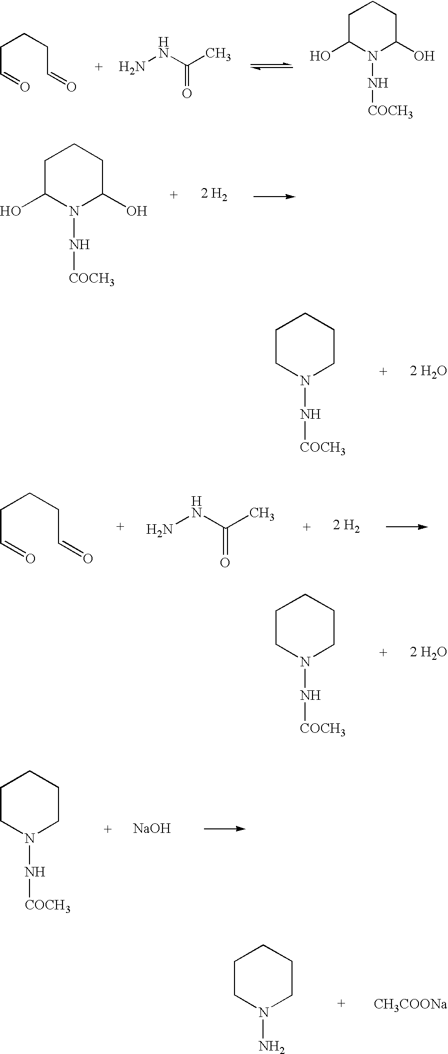

(C₁-C₈)-Alkyl is to be regarded as methyl, ethyl, n-propyl, isopropyl, n-butyl, isobutyl, sec-butyl, tert-butyl, pentyl, hexyl, heptyl or octyl together with all bonding isomers. This can be mono- or polysubstituted by (C₁-C₈)-haloalkyl, OH, halogen, NH₂.

(C₁-C₈)-Alkoxy is a (C₁-C₈)-alkyl bonded via an oxygen atom to the molecule considered.

(C₁-C₈)-Alkoxyalkyl is a (C₁-C₈)-alkyl containing an oxygen atom.

(C₃-C₈)-Cycloalkyl is understood as meaning cyclopropyl, cyclobutyl, cyclopentyl, cyclohexyl or cycloheptyl or cyclooctyl radicals.

A (C₆-C₁₈)-aryl radical is understood as meaning an aromatic radical having 6 to 18 C atoms. In particular, compounds such as phenyl, naphthyl, anthryl, phenanthryl, biphenyl radicals are included. These can be mono- or polysubstituted by (C₁-C₈)-alkoxy, (C₁-C₈)-haloalkyl, OH, halogen, NH₂, S—(C₁-C₈)-alkyl.

A (C₇-C₁₉)-aralkyl radical is a (C₆-C₁₈)-aryl radical bonded to the molecule via a (C₁-C₈)-alkyl radical.

(C₁-C₈)-Haloalkyl is a (C₁-C₈)-alkyl substituted by one or more halogen atoms. Possible halogen atoms are, in particular, chlorine and fluorine.

A (C₃-C₁₈)-heteroaryl radical is, in the context of the invention, a five-, six- or seven-membered aromatic ring system of 3 to 18 C atoms, which in the ring contains heteroatoms such as, for example, nitrogen, oxygen or sulphur. Such heteroaromatics are in particular regarded as radicals such as 1-, 2-, 3-furyl, 1-, 2-, 3-pyrrolyl, 1-, 2-, 3-thienyl, 2-, 3-, 4-pyridyl, 2-, 3-, 4-, 5-, 6-, 7-indolyl, 3-, 4-, 5-pyrazolyl, 2-, 4-, 5-imidazolyl, acridinyl, quinolinyl, phenanthridinyl, 2-, 4-, 5-, 6-pyrimidinyl. These can be mono- or polysubstituted by (C₁-C₈)-alkoxy, (C₁-C₈)-haloalkyl, OH, halogen, NH₂, NO₂, SH, S— (C₁-C₈)-alkyl.

A (C₄-C₁₉)-heteroaralkyl is understood as meaning a heteroaromatic system corresponding to the (C₇-C₁₉)-aralkyl radical.

Halogen is fluorine, chlorine, bromine, iodine.

An N-protective group is understood according to the invention as meaning the following. It can be arbitrarily chosen, provided it contains a carbonyl function and is bonded to the nitrogen via this. Such groups are familiar to the person skilled in the art (Greene, T. W., Protective Groups in Organic Synthesis, J. Wiley & Sons, 1981). Thereunder in the context of the invention he understands, in particular, a radical selected from the group: formyl, acetyl, propionyl, methoxycarbonyl, ethoxycarbonyl, tert-butoxycarbonyl, Z, Fmoc, phthaloyl.

In the context of the invention, in the presence of a number of radicals R¹ in the molecule, each can be different in the context indicated.

### EXPERIMENTAL SECTION

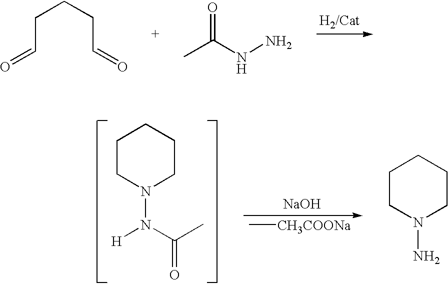

### Procedure

280.4 g (1.4 mol) of aqueous glutaraldehyde (50% by weight in water) and 115.2 g (1.4 mol) of solid acetylhydrazine are dissolved in 1400 ml of ethanol and stirred for 30 min at RT. The solution is added to a 2 l autoclave and treated with 14.0 g of a commercially available moist Pd/C (5%). A hydrogen pressure of 20 bar is set and the mixture is then hydrogenated at 80° C. After absorption of the necessary amount of hydrogen after about 4-5 h, it is cooled to RT and the autoclave is depressurized. The catalyst is filtered off. The filtrate is evaporated in vacuo in order to remove the ethanol. For complete removal of the ethanol, water is added once more and distilled off again. Under argon, 252.0 g (6.3 mol) of solid NaOH are added. The reaction mixture is refluxed for 4 h at a temperature of 128-130° C. The stirrer is switched off. The product deposits as an oily phase here. The oily phase is separated off and distilled at 100-50 mbar and a bath temperature of 90° C.

Yield: 125.7 g of product having a water content of 6.85% by weight. Corresponds to 117.1 g of 100% strength product=83.5% of theory, NMR OK.

### One-Pot Variant

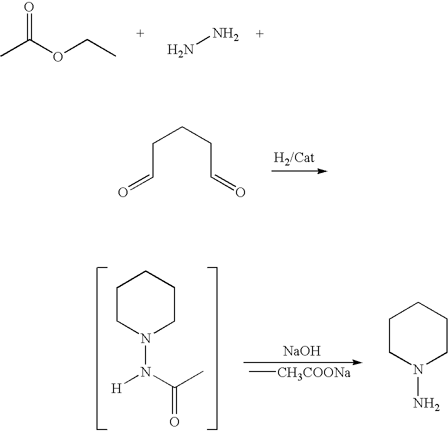

### Procedure

1 mol of ethyl acetate and 1 mol of hydrazine hydrate solution are refluxed for 10 h. The mixture is cooled to 50° C. and 1 mol of aqueous glutaraldehyde solution is added. After addition of 3 mol % Pd/C (5%), it is flushed with nitrogen, the autoclave is closed and 20 bar of hydrogen are injected. The mixture is heated to 80° C. and hydrogenated at a constant hydrogen pressure of 20 bar. The reaction is complete after 2 h. The mixture is cooled to RT, depressurized and the catalyst is filtered off. The filtrate is distilled in vacuo in order to reduce the amount of alcohol, before 4.5 mol of conc. sodium hydroxide solution are added. The mixture is refluxed for 4 h. After cooling to RT, it is worked up as described above. Yield: 75%

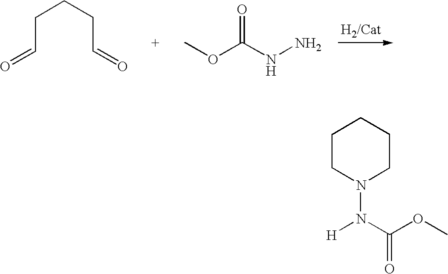

### Procedure

1081.1 g (5.4 mol) of aqueous glutaraldehyde (50% by weight in water) and 486.0 g (5.4 mol) of methyl carbazate are dissolved in 101 of ethanol. In the course of this the reaction mixture heats up to 50° C. It is stirred for 30 minutes at the same temperature. The reaction mixture is added to a 20 l autoclave. 150 g of a moist Pd/C (5%) catalyst are added and the mixture is hydrogenated at 80° C. and 20 bar of hydrogen. After 8 h, the absorption of hydrogen is complete. The autoclave is cooled to RT and depressurized. The catalyst is filtered off and the filtrate is evaporated in vacuo. The residue is dried overnight in vacuo at 50° C.

Yield: 821.8 g (98.7% of theory); 1-methoxycarbonyl-piperidine

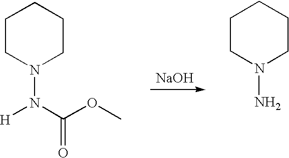

### Procedure

216 g (1.4 mol) of 1-methoxycarbonylpiperidine are hydrolysed in 500 ml of 48% strength NaOH in the course of 5 h. After switching off the stirrer, the phases are separated and the organic phase is distilled in vacuo (100-50 mbar; 85-90° C.).

Yield: 112 g (80%)

1. Process for the preparation of compounds of the general formula (I) 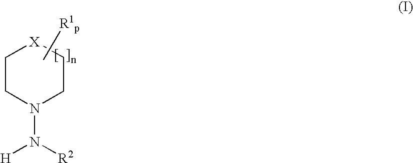
in which
n is 0 or 1,
p is 0, 1, 2, or 3,
X is CR¹R¹, O, NR², NR¹, or S,
R¹ independently of one another is H, (C₁-C₈)-alkyl, (C₁-C₈)-alkoxy, (C₁-C₈)-alkoxyalkyl, (C₃-C₈)-cycloalkyl, (C₆-C₁₈)-aryl, (C₇-C₁₉)-aralkyl, (C₃-C₁₈)-heteroaryl, (C₄-C₁₉)-heteroaralkyl, ((C₁-C₈)-alkyl)₁₋₃-(C₃-C₈)-cycloalkyl, ((C₁-C₈)-alkyl)₁₋₃-(C₆-C₁₈)-aryl, or ((C₁-C₈)-alkyl)₁₋₃-(C₃-C₁₈)-heteroaryl,
R² is H or an N-protective group which can be cleaved under acidic or basic conditions,
comprising reacting a dicarbonyl compound of the general formula (II), 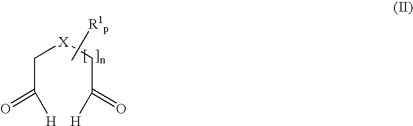
with one equivalent of the hydrazine derivative of the general formula (III) 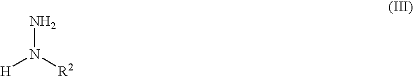
in which
R² is an N-protective group which can be cleaved under acidic or basic conditions, and subsequently hydrogenating the compound formed in the presence of a transition metal and optionally cleaving the group R² under acidic or basic conditions.

2. Process according to claim 1,
wherein
n is 1,
p is 0,
X is CH₂,
R² is acetyl or methoxycarbonyl.

3. Process according to claim 1,
wherein
the transition metal employed is a Pt- or Pd-containing compound.

4. Process according to claim 1,
wherein
the hydrogenation is carried out using H₂ gas or as a transfer hydrogenation.

5. Process according to claim 1,
wherein
an amount of transition metal complex of from 0.1-10 mol % based on the amount of compound (II) is employed.

6. Process according to claim 1,
wherein
for the cleavage of the radical R² either a solution of an inorganic acid in water or a solution of an inorganic base in water is employed.

7. Process according to claim 1,
wherein
hydrogenation is carried out at a pressure of 1-20 bar using H₂ gas.

8. Process according to claim 1,
wherein the reaction is carried out at a temperature between RT and 140° C.

9. Process according to claim 1,
wherein
the reaction of the compound of the formula (II) with the compound of the formula (III) and the hydrogenation of the compound formed is carried out as a one-pot reaction.

10. A compound of the general formula (V) 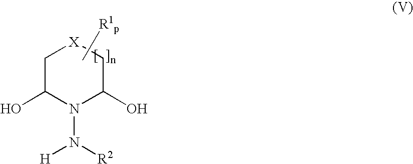
in which
m and n independently of one another is 0, 1, 2 or 3, where n and m cannot both be 0,
p is 0, 1, 2, or 3,
X is CR¹R¹, O, NR², NR¹, or S,
R¹ independently of one another is H, (C₁-C₈)-alkyl, (C₁-C₈)-alkoxy, (C₁-C₈)-alkoxyalkyl, (C₃-C₈)-cycloalkyl, (C₆-C₁₈)-aryl, (C₇-C₁₈)-aralkyl, (C₃-C₁₈)-heteroaryl, (C₄-C₁₉)-heteroaralkyl, ((C₁-C₈)-alkyl)₁₋₃-(C₃-C₈)-cycloalkyl, ((C₁-C₈)-alkyl)₁₋₃-(C₆-C₁₈)-aryl, or ((C₁-C₈)-alkyl)₁₋₃-(C₃-C₁₈)-heteroaryl,
R² is H or an N-protective group which can be cleaved under acidic or basic conditions.
